(19) United States
(12) Patent Application Publication
(10) Pub. No.: US 2024/0335211 A1
(43) Pub. Date: Oct. 10, 2024

(54) SYSTEMS, METHODS, AND DEVICES FOR REPROCESSING HARMONIC TISSUE CUTTERS

(71) Applicant: Stryker Corporation, Kalamazoo, MI (US)

(72) Inventors: Nicholas Anthony Chesley FERENTHEIL, Gilbert, AZ (US); Nathan Rampton BUTTERS, Phoenix, AZ (US); Korey Brock PETERSON, Phoenix, AZ (US); Charles Allen FORKER, Evans, GA (US)

(73) Assignee: Stryker Corporation, Kalamazoo, MI (US)

(21) Appl. No.: 18/628,663

(22) Filed: Apr. 5, 2024

Related U.S. Application Data
(60) Provisional application No. 63/494,993, filed on Apr. 7, 2023.

(51) Int. Cl. A61B 17/32; A61B 17/00

(52) U.S. Cl. CPC A61B 17/320092; A61B 2017/00017; A61B 2017/00433; A61B 2017/00477; A61B 2017/320078; A61B 2017/320094

(57) ABSTRACT

A tissue-engaging pad for a harmonic tissue cutter that comprises a shaft and a jaw pinned to the shaft, the tissue-engaging pad includes: a main body comprising a tissue-engaging surface; and a rail configured for insertion into a slot of the jaw to retain the tissue-engaging pad to the jaw, the rail comprising a stem extending from the main body opposite the tissue-engaging surface and a flange supported by the stem, wherein the flange is configured so that the rail can be pressed into the slot in a direction transverse to a longitudinal direction of the slot of the jaw.

### CROSS-REFERENCE TO RELATED APPLICATIONS

This application claims the benefit of U.S. Provisional Application No. 63/494,993, filed Apr. 7, 2023, the entire contents of which are hereby incorporated by reference herein.

### FIELD

The present disclosure relates, generally, to surgical instruments, and more particularly, to harmonic tissue cutters.

### BACKGROUND

Harmonic tissue cutters use ultrasonic mechanical vibration to cut and seal tissue during surgical procedures. These devices include an ultrasonically driven blade and a jaw that can be opened and closed to force tissue against the blade. The ultrasonic motion of the blade heats the tissue, causing collagen and elastin molecules within the tissue to melt and then fuse back together, creating a seal. The user squeezes a lever built into the handle of the harmonic tissue cutter to close the jaw and apply pressure to tissue gripped by the jaw and blade. The jaw is often provided with a tissue-engaging pad that grips and applies pressure to the tissue. The pad material and geometry are selected to withstand the amount of pressure and heat applied against the tissue by the blade for a desired tissue thickness or vessel size. The heat and force delivered by the blade during use can cause the pad to wear over time, limiting the lifespan of the harmonic tissue cutter. For this reason, harmonic tissue cutters are often considered disposable.

### SUMMARY

Devices and methods for reprocessing harmonic tissue cutters can include replacing a worn tissue-engaging pad with a replacement tissue-engaging pad that is configured for mounting to the jaw of the harmonic tissue cutter while the jaw remains mounted to a shaft of the harmonic tissue cutter. The replacement tissue-engaging pad includes a rail that fits into a slot in the jaw to retain the replacement tissue-engaging pad on the jaw. The connection between the jaw and the shaft prevents full access to the slot from its proximal entry point, and thus, the replacement tissue-engaging pad is configured for inserting the rail into the slot in an alternative fashion. For example, the rail can be configured for inserting into the slot in a direction transverse to a longitudinal direction of the slot.

According to an aspect, a tissue-engaging pad for a harmonic tissue cutter that comprises a shaft and a jaw pinned to the shaft includes: a main body comprising a tissue-engaging surface; and a rail configured for insertion into a slot of the jaw to retain the tissue-engaging pad to the jaw, the rail comprising a stem extending from the main body opposite the tissue-engaging surface and a flange supported by the stem, wherein the flange is configured so that the rail can be pressed into the slot in a direction transverse to a longitudinal direction of the slot of the jaw.

The flange may include chamfered corners. The rail may extend to at least one end face of the tissue-engaging pad. An end of the rail may be spaced from a corresponding end of the tissue-engaging pad. The end of the rail may be chamfered.

The rail may be a single continuous rail. The main body and the rail may be made of the same material. The main body and the rail may be formed as a single piece.

According to an aspect, a method of assembling a tissue-engaging pad to a jaw of a harmonic tissue cutter includes pressing a rail of the tissue-engaging pad into a slot of the jaw while the jaw is pinned to a shaft of the harmonic tissue cutter until the rail is captured by the slot so that the tissue-engaging pad is retained to the jaw.

The method may include adhering the tissue-engaging pad to the jaw with an adhesive. The adhesive may be applied after the tissue-engaging pad has been retained to the jaw.

According to an aspect, a tissue-engaging pad for a harmonic tissue cutter that comprises a shaft and a jaw pinned to the shaft includes: a main body comprising a tissue-engaging surface; a rail extending from the main body opposite the tissue-engaging surface and configured for pressing into a slot of the jaw; and at least one wedge configured to wedge into at least one slot of the rail as the rail is pressed into the slot of the jaw, thereby expanding the rail so that the rail is retained in the slot of the jaw.

The wedge may be made of a different material from the rail.

According to an aspect, a method of assembling a tissue-engaging pad to a jaw of a harmonic tissue cutter includes positioning a rail of the tissue-engaging pad in a slot of the jaw; and pressing the tissue-engaging pad onto the jaw so that at least one wedge in the slot spreads the rail until the rail is retained by the slot.

The method may include, prior to positioning the rail in the slot of the jaw, partially inserting the at least one wedge into the rail.

According to an aspect, a tissue-engaging pad for a harmonic tissue cutter that comprises a shaft and a jaw pinned to the shaft includes: a main body comprising a tissue-engaging surface; and a plurality of rail segments configured for insertion into a slot of the jaw to retain the tissue-engaging pad to the jaw, each rail segment comprising a stem extending from the main body opposite the tissue-engaging surface and a flange supported by the stem that is configured to be retained by retaining walls of the jaw, wherein the plurality of rail segments are spaced apart in a longitudinal direction of the tissue-engaging pad, wherein a length of each rail segment permits the flange of the rail segment to fit into a corresponding gap in at least one of the retaining walls so that the flange can be inserted in a lateral direction of the jaw into the slot and slid in a longitudinal direction of the jaw into engagement with the retaining walls.

A length of an end rail segment may be less than a gap between a proximal end of the retaining walls and a pin that pins the jaw to the shaft. The rail segments may be configured for retention in a T-shaped slot.

According to an aspect, a method of assembling a tissue-engaging pad to a jaw of a harmonic tissue cutter includes: positioning the tissue-engaging pad above a slot of the jaw so that rail segments of the tissue-engaging pad are aligned with gaps between retaining wall segments of the slot; lowering the tissue-engaging pad onto the jaw so that the rail segments are positioned in the slot; and sliding the tissue-engaging pad in a longitudinal direction of the slot until flanges of the rail segments are aligned with the retaining wall segments of the slot.

The method may include using adhesive to adhere the tissue-engaging pad to the jaw. The method may include machining retaining walls of the slot to form the gaps between the retaining wall segments.

According to an aspect, a tissue-engaging pad for a harmonic tissue cutter that comprises a shaft and a jaw pinned to the shaft includes a plurality of segments, each tissue-engaging pad segment comprising: a main body comprising a tissue-engaging surface; and a rail configured for insertion into a slot of the jaw to retain the tissue-engaging pad segment to the jaw, the rail comprising a stem extending from the main body opposite the tissue-engaging surface and a flange supported by the stem that is configured to be retained by the slot of the jaw, wherein the tissue-engaging pad is sized to permit the rail to fit in a space between a proximal end of the slot and a pin that pins the jaw to the shaft so that the flange can be slid into the slot from the proximal end, thereby permitting each tissue-engaging pad segment to be mounted individually to the jaw.

The plurality of segments may be formed of the same material. The main body and the rail of each tissue-engaging pad segment may be formed as a single piece. The rail may be configured for retention in a T-shaped slot.

According to an aspect, a method of assembling a tissue-engaging pad to a jaw of a harmonic tissue cutter includes: (1) positioning a first segment of the tissue-engaging pad between a proximal end of a slot of the jaw and a pin that pins the jaw to a shaft of the harmonic tissue cutter; (2) sliding a rail of the first segment into the slot of the jaw by moving the first segment in a distal direction; and (3) repeating steps 1 and 2 for at least a second segment of the tissue-engaging pad.

The method may include adhering at least the first and second segments of the tissue-engaging pad to the jaw. The method may include adhering at least the first and second segments of the tissue-engaging pad to each other. The method may include moving the first segment in the distal direction until the rail of the first segment abuts a distal end of the slot.

According to an aspect, a tissue-engaging pad for a harmonic tissue cutter that comprises a shaft and a jaw pinned to the shaft includes: a main body comprising a tissue-engaging surface; and a rail configured for insertion into a slot of the jaw to retain the tissue-engaging pad to the jaw, wherein the main body is mountable to the rail after the rail has been inserted in the slot of the jaw.

The rail may include at least one projection configured to be received in at least one receptacle of the main body for retaining the main body to the rail. The at least one projection may include a plurality or pins and the at least one receptacle comprises a plurality of sockets. The rail may be sufficiently flexible so that the rail can be slid into the slot from a proximal end of the slot while the jaw is pinned to the shaft. A material of the rail may be different than a material of the main body.

According to an aspect, a method of assembling a tissue-engaging pad to a jaw of a harmonic tissue cutter includes: inserting a rail of the tissue-engaging pad into a slot of the jaw; and after the rail has been inserted in the slot, mounting a main body of the tissue-engaging pad to the rail.

Mounting the main body to the rail may include pinning the main body and the rail together. The method may include using adhesive to mount the main body and the rail together. Inserting the rail into the slot may include bending the rail until an end of the rail fits between a proximal end of the slot and a pin that pins the jaw to a shaft of the harmonic tissue cutter, sliding the end of the rail distally into and along the slot, and unbending the rail as the rail is slid distally into the slot.

According to an aspect, a tissue-engaging pad for a harmonic tissue cutter that comprises a shaft and a jaw pinned to the shaft includes: a main body comprising a tissue-engaging surface; a rail configured for insertion into a slot of the jaw to retain the tissue-engaging pad to the jaw, the rail comprising a stem extending from the main body opposite the tissue-engaging surface and a flange supported by the stem; and a cap at an end of the rail for covering a distal end of the slot when the rail is inserted into the slot.

The cap may be configured to align with an outer surface of a distal end of the jaw. The rail may extend from the cap to an end of the main body. The main body, the rail, and the cap may be formed as a single piece.

According to an aspect, a method of assembling a tissue-engaging pad to a jaw of a harmonic tissue cutter includes inserting a rail of the tissue-engaging pad into a distal end of a slot of the jaw; and sliding the tissue-engaging pad proximally along the jaw until a cap of the tissue-engaging pad covers the distal end of the slot.

The method may include, prior to inserting the rail into the slot, machining a distal end of the jaw so that the distal end of the slot is open. The method may include adhering the tissue-engaging pad to the jaw.

According to an aspect, a method of reprocessing a harmonic tissue cutter includes reforming the tissue-engaging surface of the tissue-engaging pad by machining the tissue-engaging surface while the tissue-engaging surface is mounted to a jaw of the harmonic tissue cutter.

According to an aspect, a method of reprocessing a harmonic tissue cutter includes machining a portion of a tissue-engaging pad mounted to a jaw of the harmonic tissue cutter leaving a remaining portion mounted to the jaw of the harmonic tissue cutter; and mounting a replacement portion to the remaining portion to form a reprocessed tissue-engaging pad.

Mounting the replacement portion to the remaining portion may include mechanically affixing the replacement portion to the remaining portion. Mounting the replacement portion to the remaining portion may include adhering the replacement portion to the remaining portion.

According to an aspect, a method of reprocessing a harmonic tissue cutter includes depositing material onto a worn portion of a tissue-engaging pad of the harmonic tissue cutter while the tissue-engaging pad is mounted to a jaw of the harmonic tissue cutter to form a deposited layer; and machining the deposited layer to form a tissue-engaging surface of the tissue-engaging pad.

The material may be cold spray deposited onto the worn portion of the tissue-engaging pad.

According to an aspect, a method of reprocessing a harmonic tissue cutter includes removing a worn tissue-engaging pad from a jaw of the harmonic tissue cutter; and over-molding a replacement tissue-engaging pad onto the jaw.

According to an aspect, a method of reprocessing a harmonic tissue cutter includes machining a portion of a shaft while a jaw remains rotatably attached to the shaft to enable a tissue-engaging pad to be inserted into a slot of the jaw; and inserting the tissue-engaging pad into the slot of the jaw.

According to an aspect, a harmonic tissue cutter includes any of the tissue-engaging pads described above.

According to an aspect, a method of using a harmonic tissue cutter includes grasping tissue between a cutting jaw and any of the tissue-engaging pads described above.

It will be appreciated that any of the variations, aspects, features, and options described in view of the systems apply equally to the methods and vice versa. It will also be clear that any one or more of the above variations, aspects, features, and options can be combined.

### BRIEF DESCRIPTION OF THE FIGURES

The invention will now be described, by way of example only, with reference to the accompanying drawings, in which:

FIG. 1A illustrates an example of a harmonic tissue cutter that can be provided with a replacement tissue-engaging pad according to the principles described herein;

FIGS. 1B-1D illustrate aspects of an exemplary distal end of the harmonic tissue cutter of FIG. 1A;

FIGS. 2A and 2B illustrate an exemplary tissue-engaging pad that can replace a worn tissue-engaging pad;

FIG. 2C shows an enlarged cross section of an exemplary rail of the tissue-engaging pad of FIGS. 2A and 2B;

FIGS. 3A and 3B are cross-sections illustrating an example of pressing a rail of a tissue-engaging pad into a slot of the jaw of the harmonic tissue cutter;

FIGS. 4A and 4B are cross-sections of an exemplary blind wedge tenon configuration of a tissue-engaging pad;

FIGS. 5A and 5B are side and bottom perspective views, respectively, of an exemplary tissue-engaging pad;

FIGS. 6A-6C illustrate an exemplary configuration of a jaw configured for mounting the tissue-engaging pad of FIGS. 5A and 5B;

FIGS. 7A-7C illustrate an exemplary alternative configuration of a jaw configured for mounting the tissue-engaging pad of FIGS. 5A and 5B;

FIGS. 8A and 8B illustrate an example of a tissue-engaging pad in which the pad is divided along its longitudinal direction into separate segments;

FIG. 8C illustrates an example of a jaw to which the tissue-engaging pad can be mounted;

FIG. 9A illustrates an example of a tissue-engaging pad that includes a main body 902 and a rail that are separate components;

FIGS. 9B and 9C illustrate mounting of a rail of a tissue-engaging pad of FIG. 9A to a jaw while the jaw remains pinned to a shaft;

FIGS. 10A-10C illustrate an example of a tissue-engaging pad that has a rail that is inserted into a slot of a jaw in a proximal direction from a distal end of the slot;

FIG. 11 illustrates an example of a reprocessing technique in which a tissue-engaging pad is reformed while remaining mounted to the jaw;

FIGS. 12A and 12B illustrate a method for reforming a tissue-engaging pad 1200 that includes removing and replacing a top portion of the tissue-engaging pad, leaving a remaining portion mounted to the jaw;

FIGS. 13A and 13B illustrate reforming a tissue-engaging pad by depositing one or more layers of new material on top of the tissue-engaging pad;

FIG. 14 illustrates an example of overmolding a tissue-engaging pad onto a jaw; and

FIGS. 15A and 15B illustrate an exemplary reprocessing technique that enables a tissue-engaging pad configured the same as or similarly to a conventional tissue-engaging pad to be mounted to the jaw without modifying the jaw and without disassembling the jaw from the shaft to which it is pinned.

### DETAILED DESCRIPTION

Reference will now be made in detail to implementations and examples of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Described herein are examples of systems, methods, and devices for reprocessing harmonic tissue cutters (also often referred to as ultrasonic tissue cutters, harmonic or ultrasonic tissue dividers, and harmonic or ultrasonic clamp coagulators) by replacing a worn tissue-engaging pad with a replacement tissue engaging pad that is configured for mounting to a jaw of the harmonic tissue cutter while the jaw remains mounted to a shaft of the harmonic tissue cutter. This enables replacement of the tissue-engaging pad, without requiring time-consuming and costly disassembly and reassembly of the jaw, allowing for continued use of the harmonic tissue cutter and turning what was a disposable surgical device into a reusable surgical device.

Conventional harmonic tissue cutters include a jaw that is mounted to a shaft by a pin. A lever in the handle is used to extend and retract the shaft, which opens and closes the jaw. A conventional tissue-engaging pad is mounted to the jaw prior to the jaw being mounted to the shaft. A typical jaw includes a T-shaped slot for receiving a corresponding T-shaped rail of the tissue-engaging pad. The slot is open at its proximal end. The rail is inserted into the proximal end of the slot and pushed forward. The jaw with its mounted tissue-engaging pad is then pivotably mounted to the shaft, typically by a pin. The geometry of the jaw, shaft, and/or pin may block full access to the proximal end of the slot such that a conventional tissue-engaging pad cannot be inserted into the slot without disassembling the jaw from the shaft.

In contrast, the replacement tissue-engaging pads described herein are configured for mounting to the jaw while the jaw remains assembled to the shaft. In some examples, the rail of the tissue-engaging pad is configured for being pressed into the slot in a direction transverse to the longitudinal direction of the slot. The rail may include a chamfered flange that can be pushed through the narrowed portion of a T-shaped slot in a snap-fit configuration. Alternatively, the rail may be configured as a blind wedge tenon in which one or more wedges are forced further into the rail as the rail is pushed into the slot, with the wedges expanding the width of the rail so that it is retained by the slot.

In some examples, the rail is configured as a plurality of rail segments that can fit into corresponding openings formed in the top of the slot of the jaw. The rail segments are dropped from the top of the jaw down into the slot and slid forward into engagement with the slot. Alternatively, the entire tissue-engaging pad can be configured as a plurality of segments that can be individually inserted into the slot in sequence via a single opening formed in, for example, the proximal end of the slot.

In some examples, the rail is a separate component from the main body of the tissue-engaging pad, with each component including corresponding engagement features for connecting the two components together. The rail is inserted into the slot and then the main body is mounted to the rail. The rail can be sufficiently flexible that it can fit in a space between the proximal end of the slot and the pin that retains the jaw to the shaft while being inserted into the slot.

In some examples, the jaw and/or shaft to which the jaw is mounted may be machined to allow access to the slot so that the rail can be inserted into the slot. For example, the distal end of the jaw may be removed exposing the distal end of the slot. The tissue-engaging pad may then be slid into the slot in a proximal direction. The distal end of the tissue-engaging pad may be configured to correspond to a shape of the portion of the distal end of the jaw that was removed, forming a cap that covers the distal end of the slot. In other examples, a portion of the distal end of the shaft to which the jaw is mounted may be machined to open a direct path to the slot when the jaw is fully pivoted relative to the shaft.

In some examples, a tissue-engaging pad is refurbished by removing a worn portion of the tissue-engaging pad, leaving a remaining portion mounted to the jaw, and adding a replacement portion to the remaining portion. The replacement portion may be mounted to the remaining portion, such as via one or more mechanical fasteners and/or adhesively. The replacement portion may be deposited on the remaining portion, such as via cold spraying or over-molding. Optionally, the replacement portion may be machined, such as to achieve a finished dimension and/or to form a desired tissue-engaging surface configuration.

A harmonic tissue cutter that is reprocessed using any of the above methods and/or devices can be used during a surgical procedure, such as to grip tissue and cut and/or seal the tissue.

In the following description of the various embodiments, reference is made to the accompanying drawings, in which are shown, by way of illustration, specific embodiments that can be practiced. It is to be understood that other embodiments and examples can be practiced, and changes can be made without departing from the scope of the disclosure.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain terminology is used in this description for convenience and reference only and is not limiting. For example, the words “upwardly,” “downwardly,” “rightwardly,” and “leftwardly” refer to directions in the drawings to which reference is made. The words “inwardly” and “outwardly” refer to directions toward and away from, respectively, the geometric center of the arrangement and designated parts thereof. The words “forwardly” and “distally” refer to the direction toward the end of the arrangement that is closest to the subject, and the words “rearwardly” and “proximally” refer to the direction toward the end of the arrangement which is furthest from the subject. This terminology includes the words specifically mentioned, derivatives thereof, and words of a similar nature.

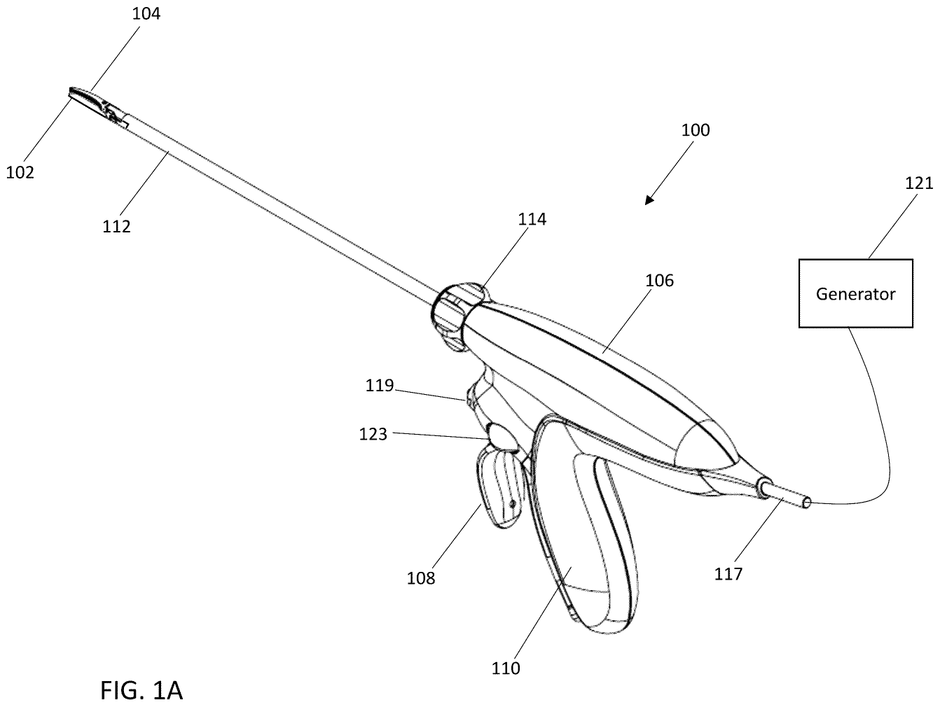

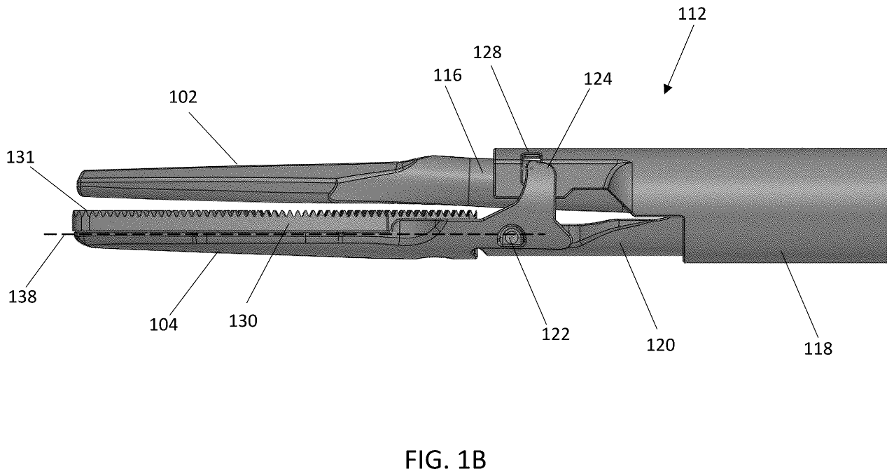

FIG. 1A illustrates an example of a harmonic tissue cutter 100 that can be provided with a replacement tissue-engaging pad according to the principles described herein. The harmonic tissue cutter 100 can be used to cut and/or seal tissue during any suitable surgical procedure, including open field procedures and minimally invasive procedures. The harmonic tissue cutter 100 includes a blade 102 and a jaw 104 that can pivot for grasping tissue between the jaw 104 and the blade 102. The blade 102 is mounted to a shaft (not shown) that extends into the main body 106. A harmonic transducer (not shown) within the main body 106 drives the shaft harmonically, causing the blade 102 to vibrate. The vibration of blade 102 can cut and seal tissue grasped between the blade 102 and jaw 104. The harmonic tissue cutter 100 may be connected via a cable 117 to a harmonic generator 121 that generates the energy to drive the transducer. A user may press an activation button 119 to activate the harmonic transducer. A selector button 123 can be used to select an amount of vibration of the blade 102.

The jaw 104 is opened and closed via a lever 108 of the handle assembly 110. The user can squeeze the lever 108 while gripping the handle assembly 110 to close the jaw 104 and put pressure on tissue grasped between the jaw 104 and the blade 102. The jaw 104 is pivotably mounted to a shaft assembly 112 that extends from the main body 106. A knob 114 may be provided to enable the user to rotate the shaft assembly 112, jaw 104, and blade 102.

FIGS. 1B-1D illustrate aspects of an exemplary distal end of the harmonic tissue cutter 100. The blade 102 extends from a blade drive shaft 116 that is slidably positioned within an outer shaft 118 of the shaft assembly 112. The jaw 104 is mounted to an inner shaft 120 that extends within the outer shaft 118. The jaw 104 is mounted to the inner shaft 120 by a pin 122 that enables the jaw 104 to pivot relative to the inner shaft 120. The jaw 104 includes a lever portion 124 that includes projections 126 (see FIG. 1C) that fit into corresponding slots 128 of the outer shaft 118. The outer shaft 118 can be moved in the proximal direction (to the right in the view of FIG. 1B) relative to the inner shaft 120 by pressing the lever 108, which through the engagement between the projections 126 and slots 128, causes the jaw 104 to close. Distal movement of the outer shaft 118 causes the jaw 104 to open.

A tissue-engaging pad 130 is mounted to the jaw 104. The tissue-engaging pad 130 includes a tissue-engaging surface 131 configured to grip and apply pressure to tissue grasped between the jaw 104 and blade 102. The tissue-engaging pad 130 can be made of a suitable polymer, such as PTFE.

FIG. 1C is a perspective view of the distal end of the harmonic tissue cutter 100 with the tissue-engaging pad 130 removed. The jaw 104 includes a slot 132 that extends in a longitudinal direction of the jaw 104. The slot 132 has an open proximal end 134 and a closed distal end 137. The slot 132 forms an opening 133 in the top surface 136 of the jaw 104.

FIG. 1D is a cross-sectional view of the distal end of the harmonic tissue cutter 100 through plane 138 of FIG. 1B showing the engagement between the tissue-engaging pad 130 and the slot 132 of the jaw 104. The only portion of the tissue-engaging pad 130 visible in the cross-section of FIG. 1D is a rail 140 that fits within the slot 132. Engagement between the rail 140 and the slot 132 retains the tissue-engaging pad 130 to the jaw 104. As shown in FIG. 1D and also in FIG. 1C, the pin 122 that pivotably mounts the jaw 104 to the inner shaft 120 is positioned just proximally of the proximal end 134 of the slot 132 preventing full access to the proximal end 134 of the slot 132. During original manufacturing of the harmonic tissue cutter 100, a tissue-engaging pad is mounted to the jaw 104 prior to the jaw 104 being mounted to the inner shaft 120 at which point the proximal end 134 of the slot 132 is fully accessible such that a distal end of the rail can be inserted into the proximal end 134 of the slot 132 and the tissue-engaging pad can be slid distally until the distal end of the rail abuts the distal end 137 of the slot 132. This pathway for mounting the tissue-engaging pad to the jaw 104 when the jaw 104 is mounted to the inner shaft 120 is blocked by the pin 122, as indicated by arrow 142. According to the principles described herein, the harmonic tissue cutter 100 can be reprocessed at least in part by replacing the original tissue-engaging pad with a tissue-engaging pad 130 that is configured for mounting to the jaw 104 while the jaw 104 remains pinned to the inner shaft 120.

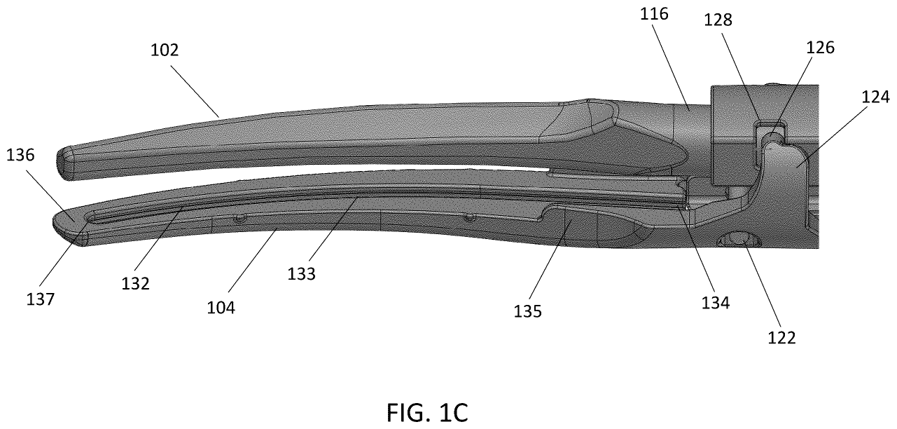

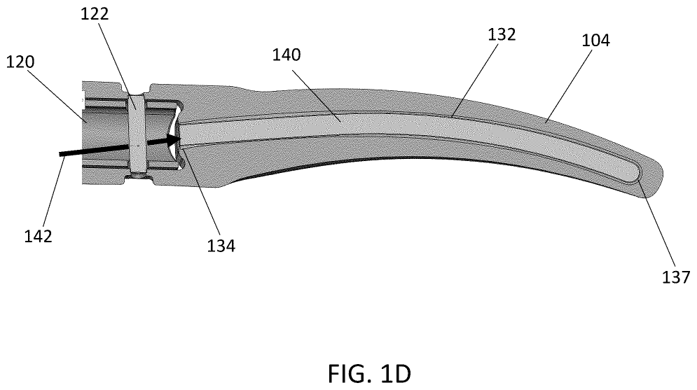

FIGS. 2A and 2B illustrate an exemplary tissue-engaging pad 200 that can be used for replacing tissue-engaging pad 130. Tissue-engaging pad 200 can be mounted to jaw 104 while jaw 104 remains pinned to inner shaft 120. Tissue-engaging pad 200 includes a rail 202 that is configured for being pressed into the slot 132 of the jaw 104 through the opening 133 in the top surface 136 of the jaw 104 in a direction that is transverse to the longitudinal direction of the slot 132. The rail 202 is configured to form a kind of snap-fit with the slot 132. The rail 202 extends on one side of a main body 204 of the tissue-engaging pad 200. The opposite side of the main body 204 includes a tissue-engaging surface 206. The tissue-engaging surface 206 may be textured to provide improved gripping of tissue. The main body 204 may taper toward a rounded distal end 208. The main body 204 can be shaped to match a shape of the original tissue-engaging pad that tissue-engaging pad 200 replaces. A proximal portion 210 of the main body 204 may be necked down to fit within side walls 135 (see FIG. 1C) of the jaw 104. The distal end 212 of the rail 202 may be spaced proximally of the distal end 208 of the main body 204. The proximal end 214 of the rail 202 may extend to the proximal end 216 of the main body 204 or may be spaced distally from the proximal end 216. The rail 202 may be a single continuous rail or may be formed of rail segments that have gaps between them. The rail 202 and main body 204 may be formed of the same material and maybe be formed as a single piece using any suitable process, such as injection molding, machining, and/or forming via an additive manufacturing process.

FIG. 2C shows an enlarged cross section of the rail 202 of the tissue-engaging pad 200. The rail 202 includes a stem 218 extending from the main body 204. The stem 218 supports a flange 220 that includes outwardly extending sides 224A,B. The flange 220 is configured for being pressed through an upper portion of the slot 132 into a lower portion of the slot 132 as will be described further below. The flange 220 extends laterally outwardly beyond the stem 218 and includes chamfers 222A,B at its lower corners. The flange 220 can extend to the distal end 212 of the rail 202 and may be chamfered at the distal end 212 of the rail 202 as illustrated in FIG. 2B. The outwardly extending sides 224A,B can extend along the full length of the rail 202 or can extend along one or more portions of the rail 202. For example, the outwardly extending sides 224A,B can be periodically interrupted so that they do not extend the full length of the rail 202, which may provide for less force being required to press the rail 202 into the slot 132 of the jaw 104. Sections of the rail 202 can include just one of the sides 224A or 224B. For example, distal and proximal portions of the rail 202 can include side 224A but not 224B and a middle portion of the rail 202 can include side 224B but not side 224A or vice versa.

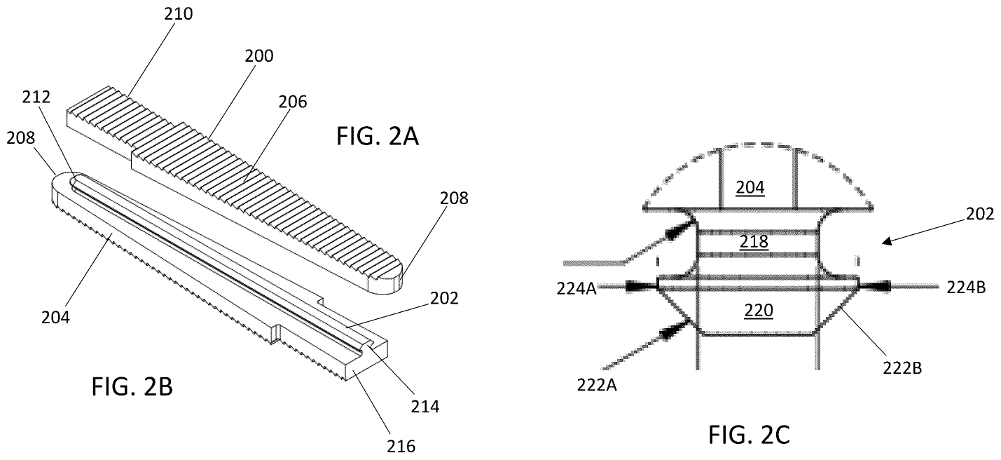

Pressing of the stem 218 into the slot 132 is illustrated in FIGS. 3A-B, which are cross-sections along a plane extending perpendicularly to the longitudinal direction of the slot 132. The slot 132 can be a T-shaped slot that includes a narrower upper portion 300 and a wider lower portion 302. The flange 220 is wider at its widest point 310 than the narrower upper portion 300 of the slot 132 and, as such, is compressed when the rail 202 is pressed downward into the slot 132 via pressure applied to the tissue-engaging surface 206 in a direction transverse (as indicated by arrow 304) to the longitudinal direction of the slot (the longitudinal direction being into the page in the view of FIGS. 3A and 3B). The chamfers 222A,B engage the upper edges 306 of the slot 132 and enable the walls of the slot 132 to compress the flange 220. The flange 220 expands back outward once it makes it past the narrower upper portion 300 of the slot 132 and into the wider lower portion 302 of the slot 132, as shown in FIG. 3B. The flange 220 is retained by retaining walls 308A,B of the jaw 104 that define the narrower upper portion 300 of the slot 132.

As noted above, the rail 202 can be pressed into the slot 132 while the jaw 104 remains mounted to the inner shaft 120. As such, reprocessing of the harmonic tissue cutter 100 using the tissue-engaging pad 200 can include removing a worn tissue-engaging pad from the jaw 104 using any suitable method or combination of methods, such as machining, cutting, breaking, prying, etc., while the jaw 104 remains mounted to the inner shaft 120, and mounting the tissue-engaging pad 200 by aligning the rail 202 with the opening 133 in the top surface 136 of the jaw 104 formed by the slot 132 and applying a force to the tissue-engaging surface 206 to push the rail 202 into the slot 132. The rail 202 can be pushed into the slot 132 in sections. For example, an installer can push the distal end 212 of the rail 202 in first and can work their way toward the proximal end 214. Optionally, adhesive may be applied to one or more surfaces of the tissue-engaging pad 200 for adhering the tissue-engaging pad 200 to the jaw 104. The adhesive may be applied prior to and/or after pushing the rail 202 into the slot 132. Optionally, the jaw 104 and inner shaft 120 may be disassembled from the main body 106 of the harmonic tissue cutter 100 and/or from the shaft assembly 112 when the tissue-engaging pad 200 is mounted to the jaw 104.

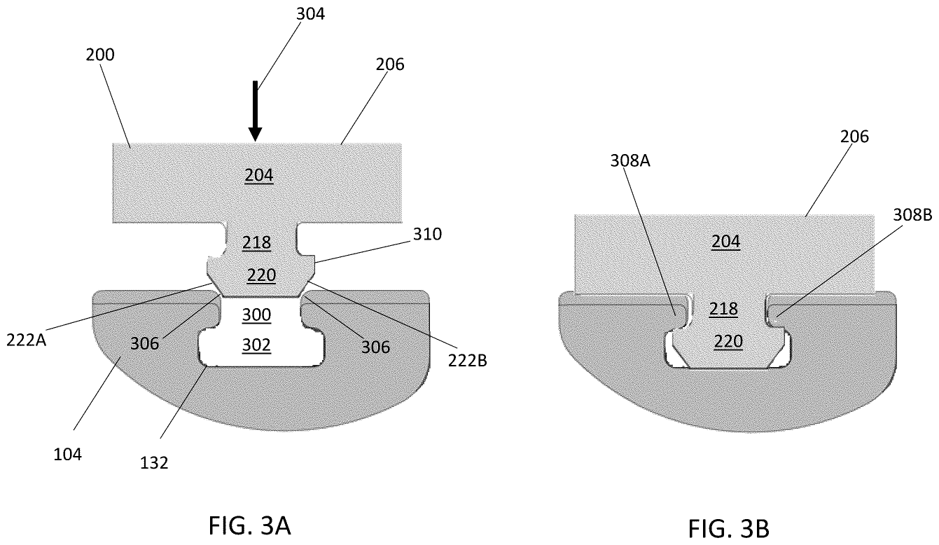

FIGS. 4A and 4B are cross-sections of an exemplary blind wedge tenon configuration of a tissue-engaging pad 400 that can be used for replacing tissue-engaging pad 130 of FIG. 1B. Like tissue-engaging pad 200 of FIG. 2, tissue-engaging pad 400 can be mounted to jaw 104 by pressing the tissue-engaging pad 400 into a slot 402 of the jaw 104 in a direction transverse to the longitudinal direction of the slot 402. Tissue-engaging pad 400 includes at least one wedge 404 (two are shown in the illustrated example) that fits into a slot 406 of the rail 408 of the tissue-engaging pad 400. As the installer pushes the rail 408 into the slot 402 of the jaw 104 by a force applied to a tissue-engaging side 414 of a main body 416 of the tissue-engaging pad 400 in the direction indicated by arrow 405, the wedge 404 bottoms out on the bottom 410 of the slot 402 of the jaw 104 and is forced further into the slot 406 of the rail 408. This causes the rail 408 to expand in a width direction until it is wider than the opening 412 of the slot 402, which retains the rail 408 in the slot 402, as shown in FIG. 4B. The slot 402 may be tapered, as illustrated, to match a tapered shape of the rail 408 when the rail 408 is fully expanded or may have any other suitable shape, including a T-shape. The wedge(s) 404 can be the same material as the rail 408 or can be a different material. In some examples, the rail 408 is made of a polymer material and the wedge 404 is made of a harder polymer material or a metal. The wedge(s) 404 can extend the full length of the rail 408 or less than the full length of the rail 408. A plurality of wedges 404 can be positioned along the length of the rail 408 and suitable intervals.

During installation of the tissue-engaging pad 400, the installer may first partially insert the wedge(s) 404 into the slot(s) 406 of the rail 408. The installer may then insert the rail 408 into the slot 402 until the wedge(s) abut the bottom 410 of the slot 402 of the jaw 104. The installer may then apply pressure to the tissue-engaging side 414 of the main body 416 of the tissue-engaging pad to force the rail 408 further into the slot 402, causing the wedge(s) 404 to wedge further into the slot(s) of the rail 408. Optionally, adhesive can be applied prior to and/or after insertion of the rail 408 into the slot 402 to adhere the rail 408 and/or the main body 416 to the jaw 104.

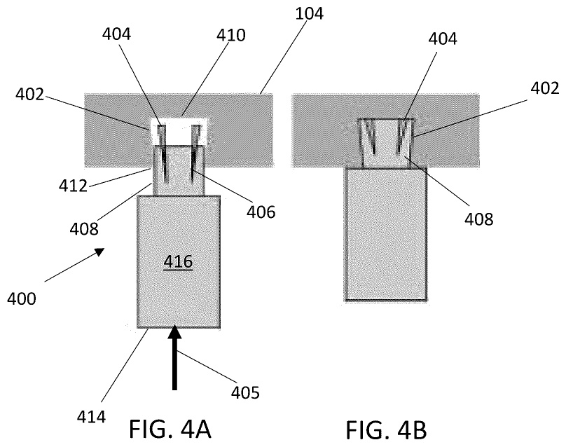

FIGS. 5A and 5B are side and bottom perspective views, respectively, of an exemplary tissue-engaging pad 500 that can be used for replacing tissue-engaging pad 130 of FIG. 1B. Tissue-engaging pad 500 is similar to tissue-engaging pad 200 of FIGS. 2A-B except that the rail is not a continuous rail but a plurality of rail segments 502 that are spaced apart on a longitudinal direction of the tissue-engaging pad 500. As described further below, the rail segments 502 are not pressed into a slot of the jaw. Rather, the rail segments 502 are configured for passing through corresponding openings in one or more retaining walls of the jaw that form the slot into which the rail segments 502 are inserted. The rail segments 502 include respective stems 504 and flanges 506 that can be shaped to correspond with a shape of a slot into which the rail segments 502 fit. Since the rail segments 502 need not be pressed into the slot, the rail segments 502 can be configured to fit closely in the slot, such as similarly to the tissue-engaging pad originally mounted to the jaw of the harmonic tissue cutter.

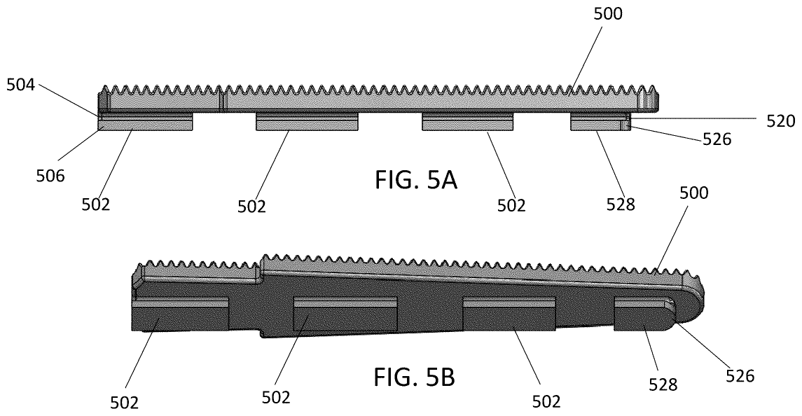

FIG. 6A-C illustrate an example of a jaw 600 configured for mounting the tissue-engaging pad 500. Jaw 104 of FIG. 1B can be configured as jaw 600. In the illustrated example, two openings 602 have been formed in a retaining wall 604 of the jaw 600 that forms one side of the slot 606 of the jaw 600. The openings 602 may be machined in the jaw 600 while the jaw 600 remains pinned to the inner shaft 120 (see FIG. 1B). The opposite retaining wall 605 is continuous. The openings 602 allow for corresponding rail segments 502 of tissue-engaging pad 500 to pass through so that the rail segments 502 can be aligned with the slot 606. Only two openings 602 are illustrated in the example of FIG. 6A for simplicity, but it will be readily understood to one of ordinary skill in the art that the number of openings can correspond to the number of rail segments of the tissue-engaging pad (e.g., four openings may be provided to accommodate the four rail segments 502 of tissue-engaging pad 500 of FIG. 5A-B). Once the rail segments 502 have passed through the openings 602 and are aligned with the slot 606, the tissue-engaging pad 500 can then be slid distally so that the flanges 506 are positioned beneath the retaining walls 604, 605, which prevents the rail segments 502 from moving laterally out of the slot 606. FIG. 6B is a cross section of jaw 600 at location 610 and FIG. 6C is a cross section of jaw 600 at location 612. The slot 606 can be a T-shaped slot like slot 132 of jaw 104 or any other suitable shape.

During mounting, the tissue-engaging pad 500 is positioned alongside the jaw 600. The tissue-engaging pad 500 is then positioned so that the rail segments 502 are located in the openings 602, which can be done either by dropping the rail segments 502 down into the openings 602 or by moving the rail segments 502 into the openings 602 from the side of the jaw 600 (or some combination of these movements). The tissue-engaging pad 500 is then moved laterally as indicated by arrow 614 until the stems 520 of the rail segments 502 are aligned with the narrower upper portion 616 of the slot 606. The tissue-engaging pad 500 is then moved distally as indicated by arrow 618 until, for example, a distal end 526 of a distal rail segment 528 abuts a distal end 620 of the slot 606. Adhesive can be used to keep the tissue-engaging pad 500 in place so that it does not slide proximally.

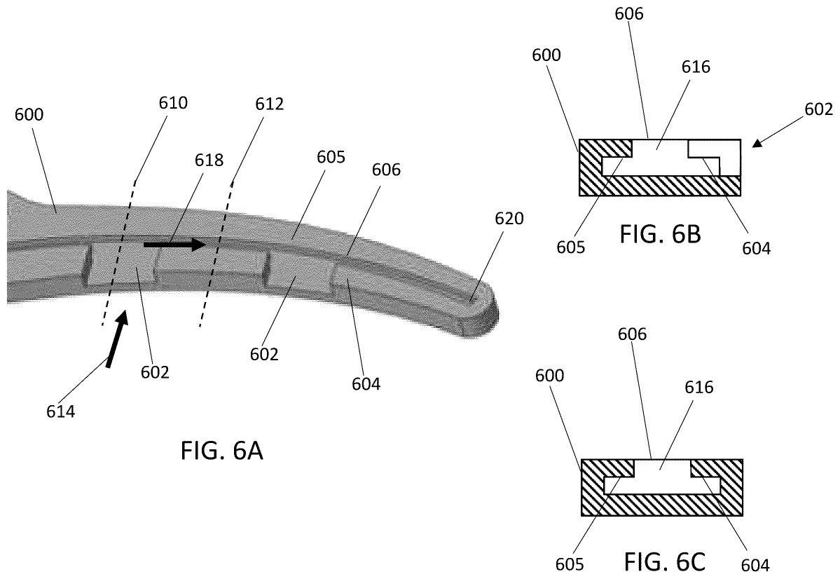

FIG. 7A-C illustrate an example of a jaw 700 configured for mounting the tissue-engaging pad 500. Jaw 104 of FIG. 1B can be configured as jaw 700. In the illustrated example, openings 702 have been formed in the retaining walls 704 and 705 of the jaw 700 on both sides of the slot 706 of the jaw 700. The openings 702 may be machined in the jaw 700 while the jaw 700 remains pinned to the inner shaft 120 (see FIG. 1B). The openings 702 allow for corresponding rail segments 502 of tissue-engaging pad 500 to pass through so that the rail segments 502 can be aligned with the slot 706. Three sets of openings 702 are illustrated in the example of FIG. 7A for simplicity, but it will be readily understood to one of ordinary skill in the art that the number of openings can correspond to the number of rail segments of the tissue-engaging pad (e.g., four sets of openings may be provided to accommodate the four rail segments 502 of tissue-engaging pad 500 of FIG. 5A-B). Once the rail segments 502 have passed through the openings 702 and are aligned with the slot 706, the tissue-engaging pad 500 can then be slid distally so that the flanges 506 are positioned beneath the retaining walls 704, 705, which prevents the rail segments 502 from moving laterally out of the slot 706. FIG. 7B is a cross section of jaw 700 at location 710 and FIG. 7C is a cross section of jaw 700 at location 712. The slot 706 can be a T-shaped slot like slot 132 of jaw 104 or any other suitable shape.

During mounting, the tissue-engaging pad 500 is positioned above the jaw 700. The tissue-engaging pad 500 is then moved downward so that the flanges 506 of the rail segments 502 are located in the openings 702 by dropping the rail segments 502 down into the openings 702 in the direction indicated by arrow 714. The tissue-engaging pad 500 continues to move downward until the flanges 506 of the rail segments 502 are aligned with the lower portion 716 of the slot 706. The tissue-engaging pad 500 is then moved distally as indicated by arrow 718 until, for example, a distal end 526 of a distal rail segment 528 abuts a distal end 720 of the slot 706. Adhesive can be used to keep the tissue-engaging pad 500 in place so that it does not slide proximally.

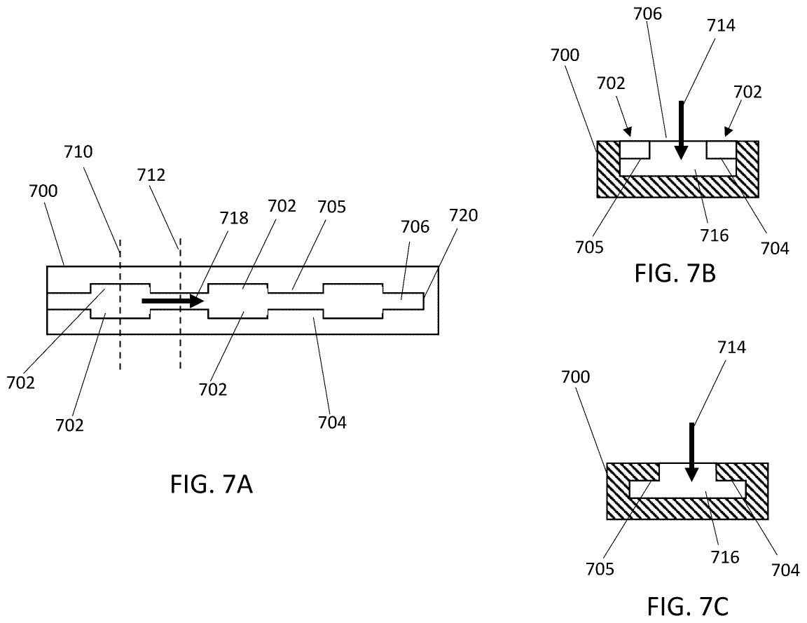

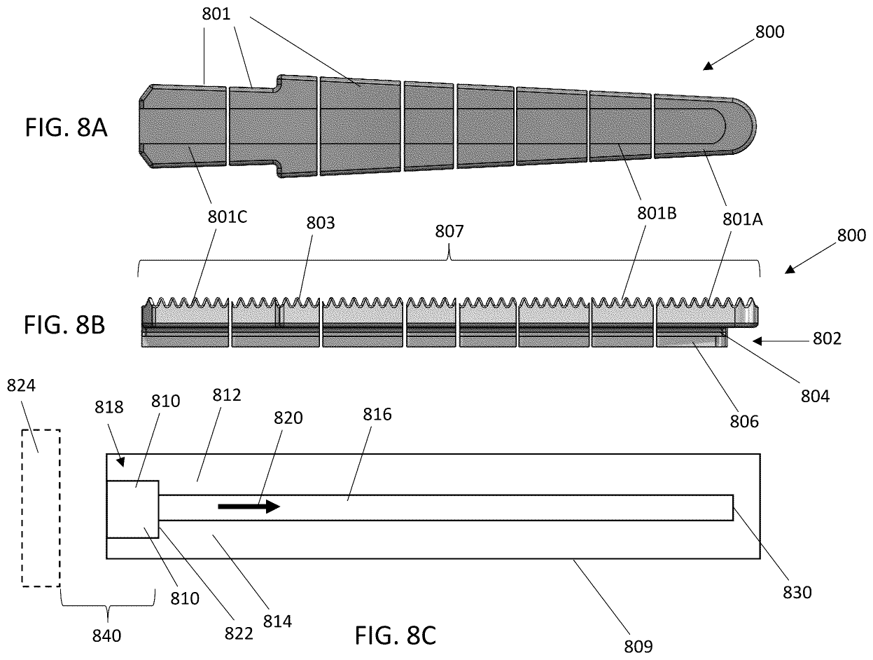

FIGS. 8A and 8B illustrate an example of a tissue-engaging pad 800 in which the pad is divided along its longitudinal direction into separate segments 801. Each segment 801 includes a tissue-engaging surface 803 and a respective rail 802 opposite the tissue-engaging surface 803 that includes a stem 804 and flange 806. The segments 801 are individually mounted to a jaw by inserting the rail 802 into a slot via one or more openings in the retaining walls of the slot and are pushed together to form a continuous or semi-continuous tissue-engaging surface 807. FIG. 8C illustrates an example of a jaw 809 to which the tissue-engaging pad 800 can be mounted. Openings 810 are provided in retaining walls 812, 814 (similar to openings 702 in walls 704, 705 of FIG. 7A) so that the flange 806 of each segment 801 can be dropped down into the slot 816 in similar fashion to the mounting of tissue-engaging pad 500 to jaw 700 described above with respect to FIGS. 7A-C. The openings 810 may be provided in a proximal end 818 of the slot 816 so that segments 801 are dropped into the slot 816 and moved distally as indicated by arrow 820. So, for example, distal segment 801A may be mounted first to the jaw 809 by inserting its rail 802 into the openings 810 and sliding the distal segment 801A distally until its rail 802 abuts a distal end 830 of the slot 816. A next segment 801B can then be inserted into the slot 816 and slid distally until it abuts the distal segment 801A. The segments 801 can continue to be mounted in this fashion until the proximal segment 801C is mounted. Adhesive may be used to adhere the segments 801 together and/or adhere the segments 801 to the jaw 809.

The openings 810 may be configured so that the space 840 between proximal ends 822 of the retaining walls 812, 814 and the pin 824 that pins the jaw 809 to the shaft (e.g., inner shaft 120 of FIG. 1B) is sufficient to accommodate the length of the rail 802 of the segments 801. The openings 810 may be machined into the jaw 809 while the jaw remains pinned to the shaft. The slot 816 may be a T-shaped slot or may have any other shape suitable for retaining the rails 802 of the segments 801. The rails 802 may have a shape (e.g., a T-shape) that matches the slot 816 or may have any other shape that is suitable for being retained by the slot 816.

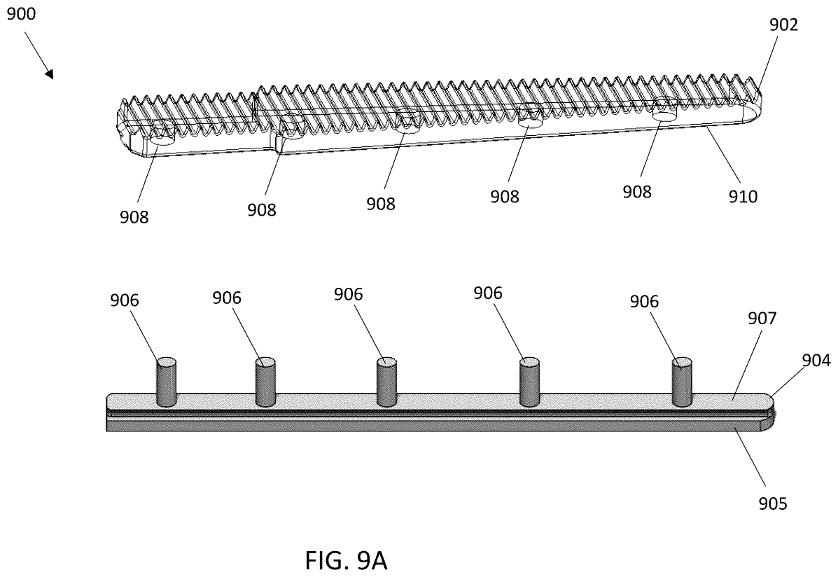

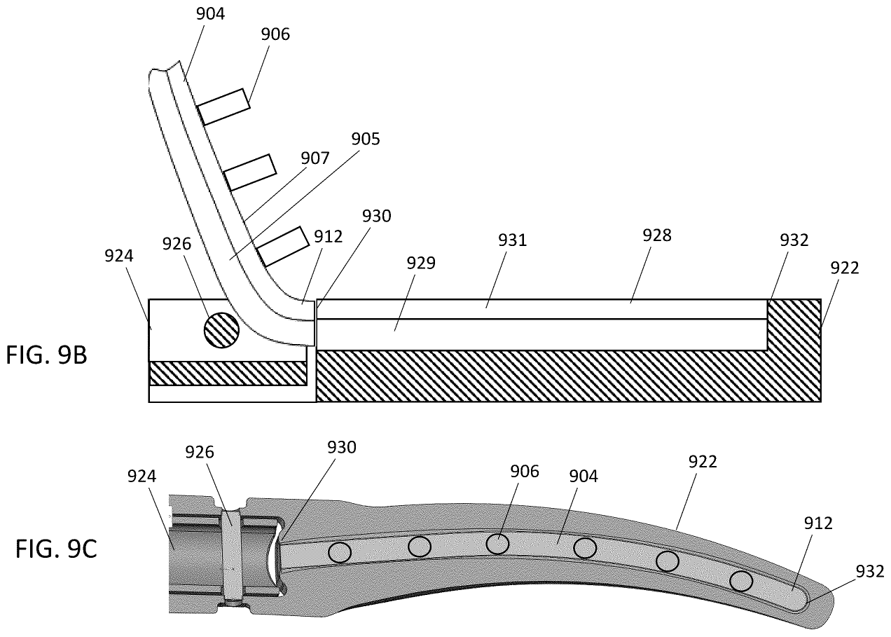

FIG. 9A illustrates an example of a tissue-engaging pad 900 that includes a main body 902 and a rail 904 that are separate components. The rail 904 is first inserted into the slot of a jaw while the jaw is mounted to a shaft and then the main body 902 is mounted to the rail 904. In the illustrated example, the rail 904 includes a plurality of projections 906 in the form of pins that are received into corresponding sockets 908 in the underside 910 of the main body 902. The projections 906 can be formed as part of the rail 904 or can be mounted to the rail 904. In some variations, the projections 906 extend from the underside 910 of the main body 902 and the sockets 908 or other types of receptacles are provided in the rail 904.

FIGS. 9B and 9C illustrate mounting of the tissue-engaging pad 900 to a jaw 922 while the jaw 922 remains pinned to a shaft 924 by a pin 926. The rail 904 is first inserted into the slot 928 in the jaw 922. The slot 928 can be a T-shaped slot or have any other suitable shape. The rail 904 can be shaped to match the shape of the slot 928. The rail 904 is configured to be sufficiently flexible that it can be bent and maneuvered between the proximal end 930 of the slot 928 and the pin 926. The distal end 912 of the rail 904 is inserted into the proximal end 930 of the slot 928 such that the flange 905 of the rail 904 fits into a lower portion 929 of the slot 928 and the stem 907 of the rail 904 fits into the upper portion 931 of the slot 928. The rail 904 is pushed in the distal direction until, for example, the distal end 912 of the rail 904 abuts the distal end 932 of the slot 928. The rail 904 unbends as it moves into the slot 928. FIG. 9C is a top view of the jaw 922 with the rail 904 fully inserted in the slot 928. The main body 902 is then pushed downward onto the rail 904 such that the projections 906 push into the sockets 908. Optionally, adhesive is used to adhere the projections 906 to the sockets 908, to adhere the rail 904 within the slot 928, and/or to adhere the main body 902 to the jaw 922. The rail 904 and main body 902 can be formed of different materials. For example, the rail 904 can be formed of a more flexible material than the main body 902.

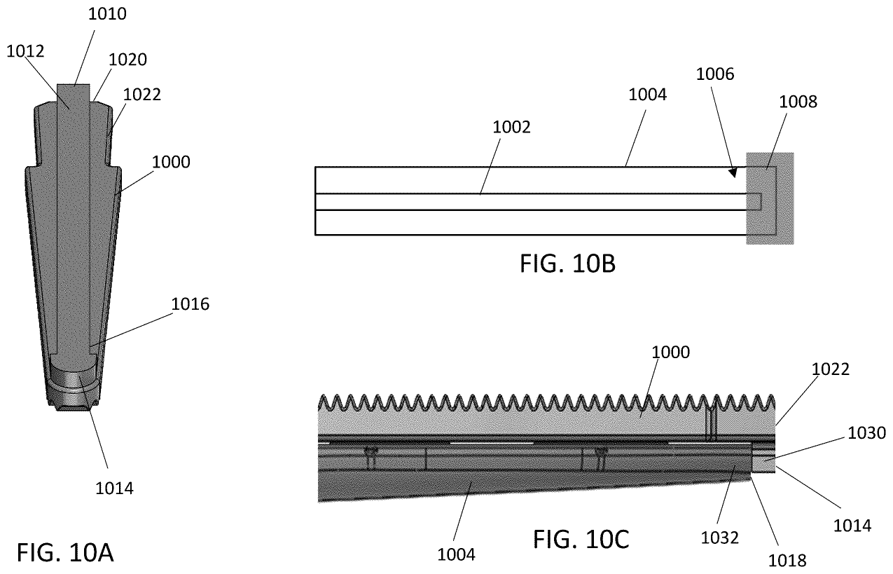

FIG. 10A-C illustrate an example of a tissue-engaging pad 1000 that has a rail 1012 that is inserted into a slot 1002 of a jaw 1004 in a proximal direction from a distal end 1006 of the slot 1002. A distal portion 1008 of the jaw 1004 may be removed such that the distal end 1006 of the slot 1002 is open. The proximal end 1010 of the rail 1012 of the tissue-engaging pad 1000 is then inserted into the distal end 1006 of the slot 1002 and the tissue-engaging pad 1000 is moved proximally. Adhesive may be applied before and/or after the insertion of the rail 1012 into the slot 1002 to adhere the tissue-engaging pad 1000 to the jaw 1004. The tissue-engaging pad 1000 may include a cap 1014 adjacent the distal end 1016 of the rail 1012 that is configured to cover the distal end 1006 of the slot 1002. The rail 1012 may extend from the cap 1014 to a proximal end 1020 of the main body 1022 of the tissue-engaging pad 1000. The cap 1014 can be shaped to match a shape of the distal portion 1008 of the jaw 1004 such that when the cap 1014 abuts the distal end 1018 of the jaw 1004, one or more outer surfaces 1030 of the cap 1014 are aligned with one or more outer surfaces 1032 of the jaw 1004. The tissue-engaging pad 1000 can be formed as a single piece or can be formed of multiple pieces that are connected together. The main body 1022 and the rail 1012 can have a similar configuration to the original tissue-engaging pad.

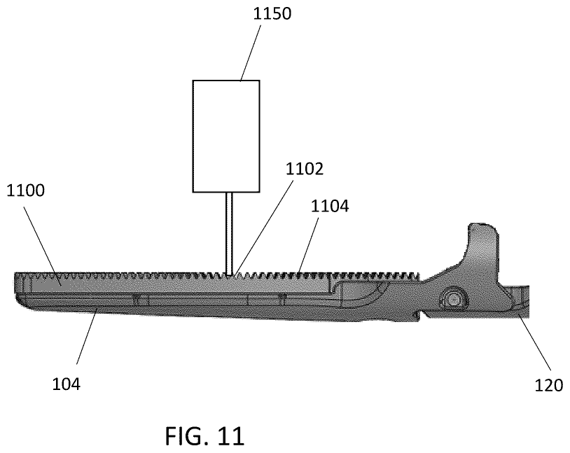

Reprocessing of a harmonic tissue cutter may not require completely removing and replacing the tissue-engaging pad. Rather, according to various examples, an existing tissue-engaging pad may be reformed while remaining mounted to, for example, jaw 104 of FIG. 1B while the jaw 104 remains pinned to the inner shaft 120. In some examples, the tissue-engaging surface of the tissue-engaging pad is reformed, such as by machining. This reprocessing technique may be used, for example, if the worn tissue-engaging pad has sufficient material thickness to enable removal of material to reform the tissue-engaging surface. An example of this reprocessing technique is illustrated in FIG. 11. Grooves 1102 in the tissue-engaging surface 1104 of a tissue-engaging pad 1100 are being machined by, for example, a milling tool 1150 while the tissue-engaging pad 1100 remains mounted to the jaw 104 and the jaw 104 remains mounted to the inner shaft 120.

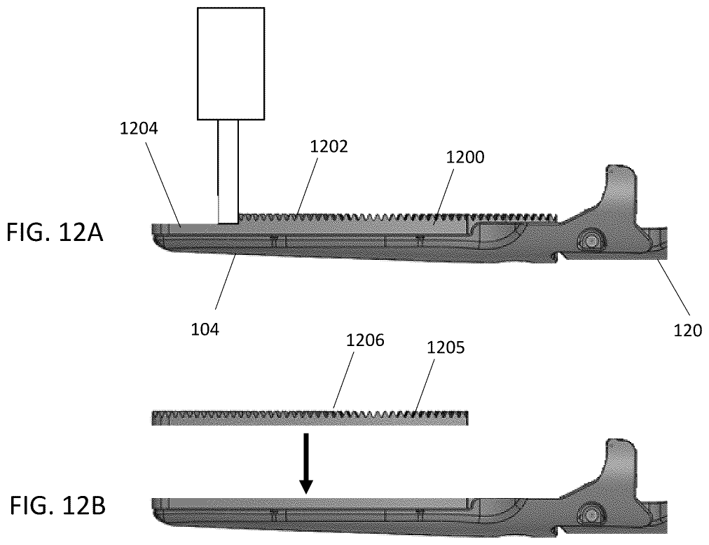

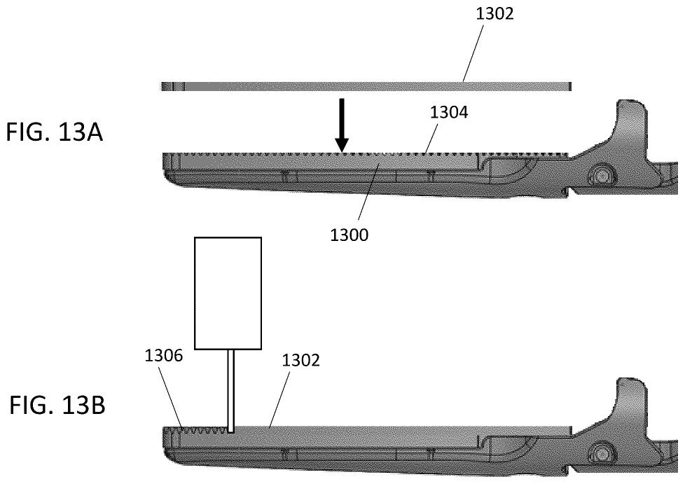

In some cases, insufficient material thickness may be available for re-machining a worn tissue-engaging surface. FIGS. 12A,B illustrate a method for reforming a tissue-engaging pad 1200 that includes removing a top portion 1202, leaving a remaining portion 1204 mounted to the jaw 104, and mounting a replacement top portion 1205 to the remaining portion 1204, such as using an adhesive or via mechanical fixation (e.g., pins or screws). The replacement top portion 1205 may have a preformed tissue-engaging surface 1206 or the tissue-engaging surface 1206 may be machined after the replacement top portion 1205 is mounted. In some examples, the replacement top portion 1205 is built up in layers, such as using any suitable additive manufacturing process (e.g., cold spraying). In an example shown in FIGS. 13A,B, one or more layers of new material 1302 is deposited on top of the worn tissue-engaging surface 1304 of a tissue-engaging pad 1300 and a reformed tissue-engaging surface 1306 is machined into the newly added material 1302.

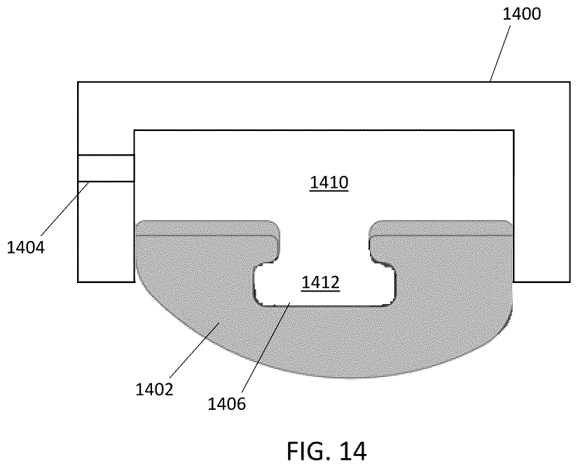

In some examples, a worn tissue-engaging pad is removed from the jaw and a replacement tissue-engaging pad is overmolded onto the jaw. FIG. 14 illustrates an example in which a mold 1400 is positioned on a jaw 1402 from which a worn tissue-engaging pad has been removed. Polymer can be injected through one or more ports 1404 into the mold 1400 to directly mold the tissue-engaging pad 1410 on the jaw. A portion of the injected polymer flows into the slot 1406 of the jaw 1402, forming a rail 1412 that retains the overmolded tissue-engaging pad 1410 to the jaw 1402. The tissue-engaging surface of the tissue-engaging pad 1410 can be formed by the mold 1400 or can be machined after the mold is removed.

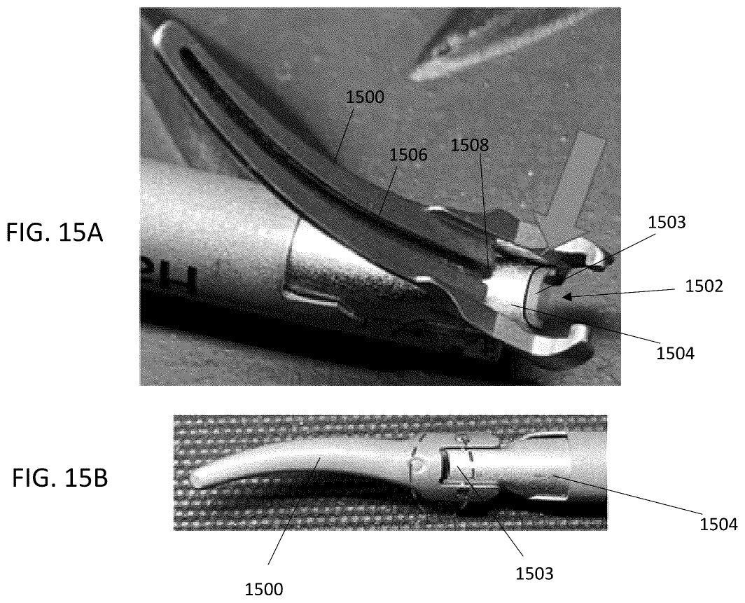

FIGS. 15A,B illustrate a reprocessing technique that enables a tissue-engaging pad configured the same as or similarly to a conventional tissue-engaging pad to be mounted to the jaw 1500 without modifying the jaw 1500 and without disassembling the jaw 1500 from the shaft 1504 to which it is pinned. The jaw 1500 may be rotated outwardly a maximum amount. A portion 1503 of the distal end 1502 of the shaft 1504 to which the jaw 1500 is mounted can be removed (i.e., via machining) to open a straight pathway to the slot 1506 of the jaw 1500. A replacement conventional tissue-engaging pad may then be mounted to the jaw 1500 by inserting a distal end of a rail of the replacement conventional tissue-engaging pad into the proximal end 1508 of the slot 1506 of the jaw 1500.

Reprocessing can also include disassembling the jaw from the rest of the harmonic tissue cutter by removing the pin that pins it to the shaft and replacing the worn tissue-engaging pad with a new tissue-engaging pad (e.g., of the same configuration), and re-assembling the jaw to the shaft.

The foregoing description, for the purpose of explanation, has been described with reference to specific examples. However, the illustrative discussions above are not intended to be exhaustive or to limit the invention to the precise forms disclosed. Many modifications and variations are possible in view of the above teachings. The examples were chosen and described in order to best explain the principles of the techniques and their practical applications. Others skilled in the art are thereby enabled to best utilize the techniques and various examples with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A tissue-engaging pad for a harmonic tissue cutter that comprises a shaft and a jaw pinned to the shaft, the tissue-engaging pad comprising:
a main body comprising a tissue-engaging surface; and
a rail configured for insertion into a slot of the jaw to retain the tissue-engaging pad to the jaw, the rail comprising a stem extending from the main body opposite the tissue-engaging surface and a flange supported by the stem, wherein the flange is configured so that the rail can be pressed into the slot in a direction transverse to a longitudinal direction of the slot of the jaw.

2. The tissue-engaging pad of claim 1, wherein the flange comprises chamfered corners.

3. The tissue-engaging pad of claim 1, wherein the rail extends to at least one end face of the tissue-engaging pad.

4. The tissue-engaging pad of claim 1, wherein an end of the rail is spaced from a corresponding end of the tissue-engaging pad.

5. The tissue-engaging pad of claim 4, wherein the end of the rail is chamfered.

6. The tissue-engaging pad of claim 1, wherein the rail is a single continuous rail.

7. The tissue-engaging pad of claim 1, wherein the main body and the rail are made of the same material.

8. The tissue-engaging pad of claim 1, wherein the main body and the rail are formed as a single piece.

9. A tissue-engaging pad for a harmonic tissue cutter that comprises a shaft and a jaw pinned to the shaft, the tissue-engaging pad comprising:
a main body comprising a tissue-engaging surface; and
a plurality of rail segments configured for insertion into a slot of the jaw to retain the tissue-engaging pad to the jaw, each rail segment comprising a stem extending from the main body opposite the tissue-engaging surface and a flange supported by the stem that is configured to be retained by retaining walls of the jaw, wherein the plurality of rail segments are spaced apart in a longitudinal direction of the tissue-engaging pad, wherein a length of each rail segment permits the flange of the rail segment to fit into a corresponding gap in at least one of the retaining walls so that the flange can be inserted in a lateral direction of the jaw into the slot and slid in a longitudinal direction of the jaw into engagement with the retaining walls.

10. The tissue-engaging pad of claim 9, wherein a length of an end rail segment is less than a gap between a proximal end of the retaining walls and a pin that pins the jaw to the shaft.

11. The tissue-engaging pad of claim 9, wherein the rail segments are configured for retention in a T-shaped slot.

12. A tissue-engaging pad for a harmonic tissue cutter that comprises a shaft and a jaw pinned to the shaft, the tissue-engaging pad comprising a plurality of segments, each tissue-engaging pad segment comprising:
a main body comprising a tissue-engaging surface; and
a rail configured for insertion into a slot of the jaw to retain the tissue-engaging pad segment to the jaw, the rail comprising a stem extending from the main body opposite the tissue-engaging surface and a flange supported by the stem that is configured to be retained by the slot of the jaw, wherein the tissue-engaging pad is sized to permit the rail to fit in a space between a proximal end of the slot and a pin that pins the jaw to the shaft so that the flange can be slid into the slot from the proximal end, thereby permitting each tissue-engaging pad segment to be mounted individually to the jaw.

13. The tissue-engaging pad of claim 12, wherein the plurality of segments are formed of the same material.

14. The tissue-engaging pad of claim 12, wherein the main body and the rail of each tissue-engaging pad segment are formed as a single piece.

15. The tissue-engaging pad of claim 12, wherein the rail is configured for retention in a T-shaped slot.

16. A tissue-engaging pad for a harmonic tissue cutter that comprises a shaft and a jaw pinned to the shaft, the tissue-engaging pad comprising:
a main body comprising a tissue-engaging surface; and
a rail configured for insertion into a slot of the jaw to retain the tissue-engaging pad to the jaw,
wherein the main body is mountable to the rail after the rail has been inserted in the slot of the jaw.

17. The tissue-engaging pad of claim 16, wherein the rail comprises at least one projection configured to be received in at least one receptacle of the main body for retaining the main body to the rail.

18. The tissue-engaging pad of claim 17, wherein the at least one projection comprises a plurality or pins and the at least one receptacle comprises a plurality of sockets.

19. The tissue-engaging pad of claim 16, wherein the rail is sufficiently flexible so that the rail can be slid into the slot from a proximal end of the slot while the jaw is pinned to the shaft.

20. The tissue-engaging pad of claim 16, wherein a material of the rail is different than a material of the main body.
